SEQUENCE LISTING (7 sequences)

SEQ ID NO: 1 - protein, 47 aa, Artificial Sequence
  Beta Defensin 3 recombinant peptide
HGGIINTLQKYYCRVRGGRCAVLSCLPKEEQIGKCSTRGRKCCRRKK

SEQ ID NO: 2 - DNA, 25 bp, Artificial Sequence
  primer
atgaggatccattatcttctgtttg

SEQ ID NO: 3 - DNA, 26 bp, Artificial Sequence
  primer
aacactctcgtcatgtttcagggttt

SEQ ID NO: 4 - DNA, 45 bp, Artificial Sequence
  primer
atgcaccaccaccaccaccaccatggaggaatcataaacacatta

SEQ ID NO: 5 - DNA, 24 bp, Artificial Sequence
  primer
ttatttctttcttcggcagcattt

SEQ ID NO: 6 - DNA, 16 bp, Artificial Sequence
  primer
gtaaaacgacggccag

SEQ ID NO: 7 - DNA, 17 bp, Artificial Sequence
  primer
caggaaacagctatgac

(19) United States
(12) Patent Application Publication
(10) Pub. No.: US 2013/0079287 A1
(43) Pub. Date: Mar. 28, 2013

(54) CAFFEOYLALPHANEOENDORPHIN PEPTIDE DERIVATIVE AND USE THEREOF AS ANTI-ITCHING AND ANTI-ATOPIC AGENTS

(71) Applicants: Dai Hyun JUNG, Gwangju (KR); Sang Hyun Moh, Yongin-si (KR); Jeong Hun Lee, Seoul (KR); Su Jung Kim, Gwangju (KR); Hyo Hyun Seo, Incheon (KR); Chang II Lim, Hwasun-gun (KR); Ji Yeon Sung, Gwangju (KR); Ae Jin Jeong, Hwasun-gun (KR)

(72) Inventors: Dai Hyun JUNG, Gwangju (KR); Sang Hyun Moh, Yongin-si (KR); Jeong Hun Lee, Seoul (KR); Su Jung Kim, Gwangju (KR); Hyo Hyun Seo, Incheon (KR); Chang II Lim, Hwasun-gun (KR); Ji Yeon Sung, Gwangju (KR); Ae Jin Jeong, Hwasun-gun (KR)

(21) Appl. No.: 13/339,773

(22) Filed: Dec. 29, 2011

(30) Foreign Application Priority Data
Sep. 22, 2011 (KR) 10-2011-0095800

(51) Int. Cl. A61K 38/08; C07K 7/08; A61Q 19/00; A61P 29/00; A61P 17/18; A61P 17/00; C07K 7/06; A61K 38/10

(52) U.S. Cl. 514/18.7; 530/328; 530/327; 514/21.5; 514/21.6; 514/18.8

(57) ABSTRACT

Provided is a caffeoylalphaneoendrophin peptide derivative, and use thereof, as an anti-itching agent and an anti-atopic agent. More specifically, provided is a caffeoyl endorphin peptide derivative, which is applicable in a cosmetic material for anti-inflammatory use, such as for an atopic dermatitis treatment, and the like. The caffeoyl endorphin peptide derivative is safe for skin, has resistance to degradation by peptidases, and the like, and also has an excellent stability with respect to temperature change, and the like.

### CROSS-REFERENCE TO RELATED PATENT APPLICATION

This application claims the benefit of Korean Patent Application No. 10-2011-0095800, filed on Sep. 22, 2011, in the Korean Intellectual Property Office, the disclosure of which is incorporated herein in its entirety by reference.

### BACKGROUND OF THE INVENTION

1. Field of the Invention

The present invention relates to a caffeoylalphaneoendorphin peptide derivative, and use thereof as anti-itching and anti-atopic agents. More specifically, the present invention relates to a caffeoyl endorphin peptide derivative, which is applicable to cosmetic materials for anti-inflammatory use, such as an atopic dermatitis treatment, and the like, because it is safe for skin; has resistance of degradation by peptidase, and the like, when applying over the skin at the same time; and also has an excellent stability in respect of temperature change, and the like, and use thereof.

2. Description of the Related Art

Skin protects a human body while directly contacting the external environment, and has biochemical and physical functions, so that it is very important tissue. A human skin surface stays moist, is smooth and shiny because the stratum corneum is covered with the sebaceous membrane that is naturally well-mixed with water secreted from the sweat glands and sebum secreted from the sebaceous glands. However, when the water content inside the skin is decreased, the dead skin is exfoliated, and foreign substances are penetrated so that all sorts of inflammations and allergies can be easily caused. An inflammation is caused for a series of protection purposes in order to minimize the reaction when cell or tissue receive damage due to any reasons and recover the damaged area. The inflammation includes skin acne, an atopic dermatitis, and the like.

Especially, in the case of the atopic dermatitis, an accurate pathogical physiology is still not understood completely, but it is considered that immunological and environmental factors along with genetic factors are related and specifically, many researches about the immunological aspect are being performed. An exogenous atopic dermatitis that is occupied most of the atopic dermatitis is caused by an immune mechanism related to IgE, and there are many reports that the exogenous atopic dermatitis is related to delayed-type immune responses due to T-cell abnormality, not immediate-type immune responses about a specific allergen. Because a main skin symptom of the atopic dermatitis is eczematous lesion, but not erythema and wheal identified by Prick Skin Test, it is still a major controversial issue to evaluate a direct correlation of the atopic dermatitis only with a result of Prick Skin Test that is the immediate-type immune responses test. Since it turned out that cell-mediated immune system disorder in a patient suffered with the atopic dermatitis is related to IgE increase, it is reported that the atopic dermatitis relates to the delayed-type immune response by T-cell disorder, rather than the immediate-type immune response against a specific allergen. It is known that about 80% of the patients suffered from the atopic dermatitis cause a cell-mediated immune disorder, in which the patients show that sensitivity about infectious diseases of skin caused by virus, dermatomyces, and the like is increased and sensitivity about a contact allergen is reduced. In the past, T cell maturation deficiency theory, a decline in CD8+ suppression T cell numbers, and the like were reported, but recently, it is reported that a role of CD4+ T cell is more important. That is, it is reported that T cell that secretes IL-4 that induces IgE generation, IL-5 that stimulate B cell to generate IgE, IL-6 that proliferates IgE generation, and the like, is Th2 among CD4+ T cell; it is confirmed that an antigen-specific T cell clone isolated from skin lesions and peripheral blood of the patients suffered from the atopic dermatitis is Th2, and also IL-4 and IL-13 isolated from these cells stimulate IgE generation and IL-5 increases basophil responses. According to the recent reports, it is reported that an immune response depends on the atopic dermatitis condition, and also an acute atopic dermatitis relates to Th2 and a chronic atopic dermatitis relates to Th1. IL 4 and IL 13 secreted from Th2 do important works in the early days of the atopic dermatitis, and when started scratching due to an itching, it moves to Th1 cell.

First of all, strong anti-inflammatory and anti-oxidative components are essential in order to protect a skin structure damaged from the atopic dermatitis and recover the damaged skin. For this reason, the inventors present various endorphins nucleopeptides having anti-itching and anti-inflammatory effects as a precedence study in Korean Registration Patent 10-1057938 (Anti-inflammatory Cosmetic Composition for Wound Healing and Tissue Regeneration). However, a biological stability of peptide is limited for applying over the skin because the peptide is very weak in the action of peptidase or proteolysis in vivo.

Meanwhile, it is known that phytochemicals, such as caffeic acid have strong anti-inflammatory, anti-cancer, and anti-oxidative functions. However, caffeic acid is also a non-soluble material so that it is limited for applying over the skin, and after dissolving it in a small quantity of water, it has a problem that it is easily oxidized and decomposed in a solution so that it is limited for using it.

### SUMMARY OF THE INVENTION

The inventors completed the present invention by confirming an effective application to a skin through synthesizing one new material by chemically conjugating with endorphin peptides that is a kind of neuropeptide in order to apply the above-mentioned caffeic acid to a skin, in which the new peptide derivative has functions of skin inflammatory alleviation, treatment, and prevention, such as an atopic dermatitis treatment, and the like, and also can resolve the problems of a physical stability, a biological stability, and the like, such as, non-soluble property, application limits of the caffeic acid and endorphin peptide, respectively to a skin, and the like.

An object of the present invention is to provide a peptide derivative represented by the following Chemical Formula 1, which is new material having anti-inflammatory and anti-oxidative effects for treating an atopic dermatitis:

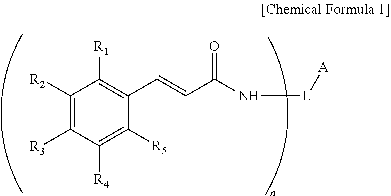

where,

R₁, R₄, and R₅ are independently hydrogen, hydroxy, C₁-C₆ alkyl group, C₁-C₆ alkoxy group, cyano, halo, or amino, respectively;

R₂ and R₃ are hydroxy;

n is 1;

L is not existed, or is a binding group; and

A is peptide or fragment thereof having sequences of Tyr-Gly-Gly-Phe-Leu-Arg-Lys-Tyr-Pro-Lys (YGGFLRKYPK) or Tyr-Gly-Gly-Phe-Leu-Arg-Arg-Ile-Arg-Pro-Lys-Leu-Lys (YGGFLRRIRPKLK).

Also, another object of the present invention is to provide an anti-inflammatory or an anti-oxidative composition including a caffeoyl peptide derivative represented by the above Chemical Formula 1.

Also, another object of the present invention is to provide a composition including the caffeoyl peptide derivative represented by the above Chemical Formula 1 for treating or preventing an atopic dermatitis.

Also, another object of the present invention is to provide a composition including the caffeoyl peptide derivative represented by the above Chemical Formula 1 for preventing or alleviating a skin itching.

Also, another object of the present invention is to provide a composition including the caffeoyl peptide derivative represented by the above Chemical Formula 1 for suppressing or reducing an expression of inflammatory factor, such as IL-4, IL-5, IL-13, IL-31, IL-10, TNFα, or IL-17.

Also, another object of the present invention is to provide a method for treating or preventing an atopic dermatitis, or alleviating, treating, or preventing an inflammation by using the caffeoyl peptide derivative represented by the above Chemical Formula 1.

Also, another object of the present invention is to provide a method for preventing or alleviating the skin itching by using the caffeoyl peptide derivative represented by the above Chemical Formula 1.

Also, another object of the present invention is to provide a method for suppressing or reducing an expression of inflammatory factor, such as IL-4, IL-5, IL-13, IL-31, IL-10, TNFα, or IL-17 by using the caffeoyl peptide derivative represented by the above Chemical Formula 1.

Also, another object of the present invention is to provide use of the caffeoyl peptide derivative represented by the above Chemical Formula 1 for anti-inflammatory, anti-oxidative, anti-atopic dermatitis, or anti-itching function.

The technical objects of the present invention are not limited to those described above, and it will be apparent to those of ordinary skill in the art from the following description that the present invention includes other technical objects not specifically mentioned herein.

According to an aspect of the present invention, the present invention provides a peptide derivative represented by the following Chemical Formula 1:

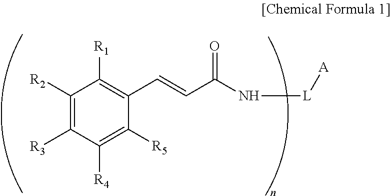

where,

R₁, R₄, and R₅ are independently hydrogen, hydroxy, C₁-C₆ alkyl group, C₁-C₆ alkoxy group, cyano, halo, or amino, respectively;

R₂ and R₃ are hydroxy;

n is 1;

L is not existed, or is a binding group; and

A is peptide or fragment thereof having sequences of Tyr-Gly-Gly-Phe-Leu-Arg-Lys-Tyr-Pro-Lys (YGGFLRKYPK) or Tyr-Gly-Gly-Phe-Leu-Arg-Arg-Ile-Arg-Pro-Lys-Leu-Lys (YGGFLRRIRPKLK).

The present invention provides an anti-inflammatory or an anti-oxidative composition, such as cosmetics and pharmaceutical composition, in which the composition includes the caffeoyl peptide derivative represented by the above Chemical Formula 1.

In addition, the present invention provides a composition for treating or preventing the atopic dermatitis, such as cosmetics and pharmaceutical composition, in which the composition includes the caffeoyl peptide derivative represented by the above Chemical Formula 1.

In addition, the present invention provides a composition for preventing or alleviating the skin itching, in which the composition includes the caffeoyl peptide derivative represented by the above Chemical Formula 1.

In addition, the present invention provides a composition for reducing or suppressing the expression of inflammatory factor, such as IL-4, IL-5, IL-13, IL-31, IL-10, TNFα, or IL-17, in which the composition includes the caffeoyl peptide derivative represented by the above Chemical Formula 1.

In addition, the present invention provides a method for treating or preventing the atopic dermatitis, or a method for alleviating, treating, or preventing the inflammation by using the caffeoyl peptide derivative represented by the above Chemical Formula 1.

In addition, the present invention provides a method for preventing or alleviating the skin itching by using the caffeoyl peptide derivative represented by the above Chemical Formula 1.

In addition, the present invention provides a method for suppressing or reducing the expression of inflammatory factor, such as IL-4, IL-5, IL-13, IL-31, IL-10, TNFα, or IL-17 by using the caffeoyl peptide derivative represented by the above Chemical Formula 1.

### BRIEF DESCRIPTION OF THE DRAWINGS

The above and other features and advantages of the present invention will become more apparent by describing in detail exemplary embodiments thereof with reference to the attached drawings in which:

FIG. 1 is a diagram illustrating a procedure for synthesizing caffeoylalphaneoendorphin peptide derivative by using Solid Phase Synthsis;

FIG. 2 is a test result for confirming an increase of resistance against acidification of aqueous solution of caffeoylalphaneoendorphin peptide derivative (CA (left): Caffeic Acid, CA-NP (right): Caffeoylalphaneoendorphin);

FIG. 3 is a graph of test result showing an increase of stability of caffeoylalphaneo endorphin to peptidase derived from a skin cell and keratinocyte (Left bar: Alphaneoendorphin, Right bar: Caffeoylalphaneoendorphin peptide derivative).

FIG. 4 is a diagram showing different sites of animal model that were applied with test materials for confirming Skin Irritation Safety Test;

FIG. 5 is results of Skin Irritation Safety Test under the conditions of 24 hours after applying (Up) and 72 hours after applying (Down) of caffeoylalphaneoendorphin peptide derivative;

FIG. 6 is a photograph showing atopic dermatitis inducement result of animal model with inducible atopic skin dermatitis (Normal: normality, AD: Atopic Dermatitis Inducement Model);

FIG. 7 is a photograph result for confirming atopic dermatitis improvement effect of caffeoylalphaneoendorphin peptide derivative through inducible atopic skin dermatitis animal model (Cont.: 70% EtOH, pep. 400: Caffeoylalphaneoendorphin peptide derivative 400 ug/each ear, pep. 800: Caffeoylalphaneoendorphin 800 ug/each ear);

FIG. 8 is results showing atopic dermatitis improvement effects of caffeoylalphaneoendorphin peptide derivative through H&E Staining Method (Cont.: 70% EtOH, pep. 400: Caffeoylalphaneoendorphin peptide derivative 400 ug/each ear, pep. 800: Caffeoylalphaneoendorphin 800 ug/each ear);

FIG. 9 is a graph showing results of effects of caffeoylalphaneoendorphin peptide derivative treatment on IgE Concentration in serum (Up) and IgE Concentration in cell culture medium (Down) (Cont.: 70% EtOH, pep. 400: Caffeoylalphaneoendorphin peptide derivative 400 ug/each ear, pep. 800: Caffeoylalphaneoendorphin 800 ug/each ear);

FIG. 10 is a graph showing decrease effects of atopic dermatitis inducible cytokines, IL-4, IL-5, and IL-13 in lymphnode CD4+ T cell of atopic dermatitis inducible animal model by treating caffeoylalphaneoendorphin (pep. 400: Caffeoylalphaneoendorphin peptide derivative 400 ug/each ear, pep. 800: Caffeoylalphaneoendorphin 800 ug/each ear);

FIG. 11 is a graph showing decrease effects of atopic dermatitis inducible cytokines, IL-4, IL-5, and IL-13 in the whole ear cells of atopic dermatitis inducible animal model by treating caffeoylalphaneoendorphin (Cont.: 70% EtOH, pep. 400: Caffeoylalphaneoendorphin peptide derivative 400 ug/each ear, pep. 800: Caffeoylalphaneoendorphin 800 ug/each ear); and

FIG. 12 is a graph showing decrease effects of ear scratching of atopic dermatitis inducible animal model by treating caffeoylalphaneoendorphin (Cont.: 70% EtOH, pep. 400: Caffeoylalphaneoendorphin peptide derivative 400 ug/each ear, pep. 800: Caffeoylalphaneoendorphin 800 ug/each ear).

### DETAILED DESCRIPTION OF THE INVENTION

Hereinafter, preferred embodiments will be described in more detail with reference to the accompanying drawings. Moreover, detailed descriptions of well-known functions or configurations will be omitted in order not to unnecessarily obscure the focus of the present invention.

For an aspect of the present invention, the present invention relates to a peptide derivative represented by the following Chemical Formula 1:

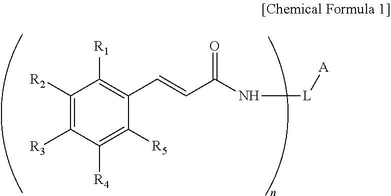

where,

R₁, R₄, and R₅ are independently hydrogen, hydroxy, C₁-C₆ alkyl group, C₁-C₆ alkoxy group, cyano, halo, or amino, respectively;

R₂ and R₃ are hydroxy;

n is 1;

L is not existed, or is a binding group; and

A is peptide or fragment thereof having sequences of Tyr-Gly-Gly-Phe-Leu-Arg-Lys-Tyr-Pro-Lys (YGGFLRKYPK) or Tyr-Gly-Gly-Phe-Leu-Arg-Arg-Ile-Arg-Pro-Lys-Leu-Lys (YGGFLRRIRPKLK).

For the present specification, alkyl group of C₁-C₆ means linear type or branched type hydrocarbon consisting of 1 to 6 carbon number, and for example includes methyl, ethyl, n-propyl, i-propyl, n-butyl, and the like, but is not limited thereto.

Alkoxy group of C₁-C₆ means linear type or branched type alkoxy group consisting of 1 to 6 carbon number, and for example includes methoxy, ethoxy, n-propanoxy, and the like, but is not limited thereto.

A general rule to name peptide for the present specification is based on three characters or one character amino acid code application, except that it is specifically indicated. In other words, a central part of amino acid structure is displayed with three characters code (for example, Ala, Lys) or one character code (for example A, K), and D-dimensional type is displayed by writing “D-” before three characters code (for example, D-Ala, D-Lys), and when not specifically indicating, L-dimensional type is assumed.

The amino acid residues that consist of peptide are possible to be both of natural or non-natural amino acid residues.

The present invention developed new material synthesized by inserting a phytochemical caffeic acid with strong anti-inflammatory and anti-oxidative effects as a binder into a part of endorphin peptides that is a ligand of k-type opioid receptor (KOR) with an offset function of activated m-type opioid receptor (MOR) as a factor for improving or alleviating the atopic dermatitis by relieving the itching of the atopic dermatitis. For example, there are alphaneoendorphin and dynorphin, and dynorphin is endorphin peptides among neuropeptides, and functions as ligand of k-type opioid receptor (KOR).

The endorphin peptide derivative according to the present invention may be produced by firstly synthesizing endorphin peptide, and then coupling reacting with caffeic acid.

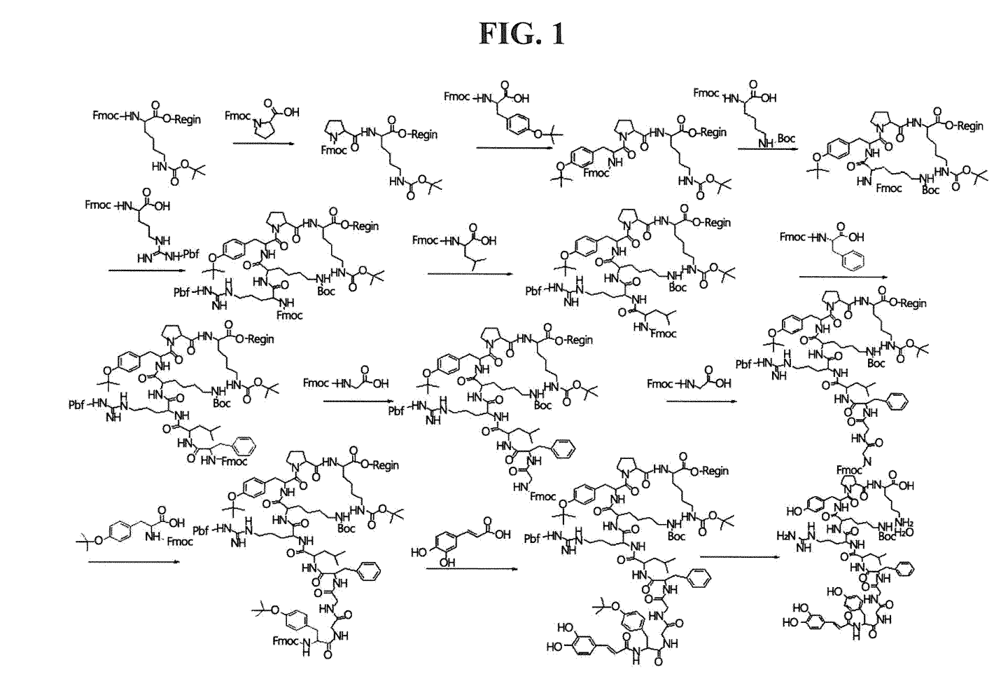

Specifically, the present invention developed a synthesis method based on solid-phase synthesis platform for high efficiency production of caffeoyl endorphin peptide derivative according to the present invention (see FIG. 1), and the peptide is a material produced by bonding two or more amino acids in a chemically bonding type that is peptide bond, and recently Solid-Phase Peptide Synthesis (SPPS) that was invented by Bruce Merrifield in 1963 is mostly used. The present invention also developed caffeoyl endorphin peptide derivative by using the solid-phase peptide synthesis. To produce the caffeoyl endorphin peptide derivative, endorphin peptide is firstly synthesized by using amino acid with a protecting group on insoluble polystyrene resin, and then the endorphin peptide is not removed on a synthesizing support, but after chemically and directly synthesizing the caffeic acid, non-bonding caffeic acid is removed from reactant to develop a platform.

For the present invention, peptide may be alphaneoendorphin or dynorphin, and amino acid sequence, A may be peptide or fragment thereof having the sequences of Tyr-Gly-Gly-Phe-Leu-Arg-Lys-Tyr-Pro-Lys (YGGFLRKYPK) or Tyr-Gly-Gly-Phe-Leu-Arg-Arg-Ile-Arg-Pro-Lys-Leu-Lys (YGGFLRRIRPKLK).

At this time, the fragment means a part of whole sequence of the peptide, and includes one including the amino acid sequence of 1, 2, 3, 4, 5, 6, 7, 8, 9, or 10 or more among the sequences, or one substituted a part of sequences with other amino acids.

The endorphin peptide may be made in low molecular weight by treating with protease after extracting protein in vivo, or produced by using genetic recombination and protein expression system, and preferably produced by using chemical synthesis using a peptide synthesizer, and the like.

For example, the peptide derivative according to the present invention may be produced by including the following steps:

(1) obtaining NH₂-protected peptide-resin by general solid phase peptide synthesis (SPPS);

(2) reacting the obtained NH₂-protected peptide-resin with caffeic acid; and

(3) removing the resin.

When a functional group is presented on side chain of amino acid residues that consist the desired peptide, the peptide may be synthesized by using the functional group-protected amino acid in the above step (1), and then the protecting group bonding to the functional group is removed in the above step (3).

One specific example of procedure for producing the peptide derivative according to the present invention using the functional group-protected amino acid is simplify shown in the following Reaction Formula 1:

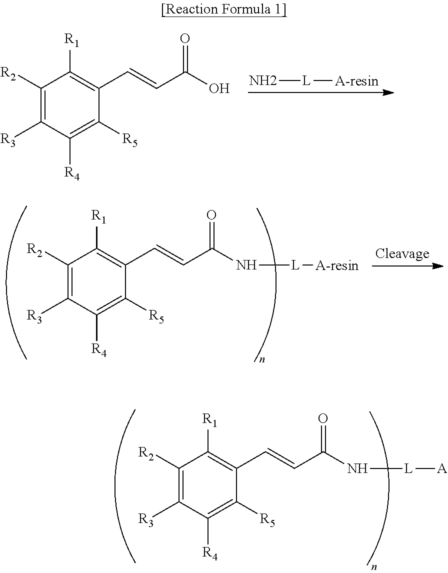

For another aspect of the present invention, the present invention relates to a cosmetic composition including the peptide derivative represented by the above Chemical Formula 1 according to the present invention, and more specifically, to a cosmetic composition with anti-oxidative and anti-inflammatory effects. In addition, the present invention relates a cosmetic composition that can be used for treating or preventing anti-inflammatory, inter alia, the atopic dermatitis, because the cosmetic composition has the effects for treating or preventing anti-inflammatory, inter alia, the atopic dermatitis. In addition, it may be the composition for anti-inflammatory treatment or prevention.

For another aspect of the present invention, the present invention relates to the composition for preventing or alleviating the skin itching, in which the composition includes the peptide derivative represented by the above Chemical Formula 1 according to the present invention.

The composition may be produced through various methods, such as a formulation method using proper process that is known in the prior art, the content of the peptide derivative represented by the above Chemical Formula 1 according to the present invention is not specifically limited, and any content numbers may be used if it is within the range that shows an effectiveness.

For another aspect of the present invention, the present invention relates to the composition for suppressing or reducing the expression of inflammatory factor, such as IL-4, IL-5, IL-13, IL-31, IL-10, TNFα, or IL-17, in which the composition includes the caffeoyl peptide derivative represented by the above Chemical Formula 1.

In addition, the present invention provides a method for alleviating, treating, or preventing an inflammation, or a method for treating or preventing the atopic dermatitis by using the caffeoyl peptide derivative represented by the above Chemical Formula 1.

In addition, the present invention provides a method for preventing or alleviating a skin itching by using the caffeoyl peptide derivative represented by the above Chemical Formula 1.

In addition, the present invention relates to a method for suppressing or reducing the expression of inflammatory factor, such as IL-4, IL-5, IL-13, IL-31, IL-10, TNFα, or IL-17, by using the caffeoyl peptide derivative represented by the above Chemical Formula 1.

The specific step of the above methods includes administrating the peptide derivative itself to a subject by using a proper administrating method (such as, applying to a skin, oral administration, intravenous administration, and the like) that is known in the prior art.

The content of the endorphin peptide derivative, which is included in the cosmetic composition of the present invention, according to the present invention may be preferably 0.005 wt % to 1 wt % based on an aqueous solution weight of 50 ppm to 10,000 ppm.

The cosmetic composition according to the present invention includes components that are generally used for the cosmetic composition, for example, a general adjurvants, such as stabilizer, solubilizer, vitamins, pigments and flavorings, and carrier, in addition to the peptide derivative according to the present invention as an effective component.

The cosmetic composition according to the present invention may be produced in any formulations that are generally used in the field, and for example, may be formulated in solution, suspension, turbid fluid, paste, gel, cream, powder, spray, and the like.

The carrier may be included in about 1 wt % to about 99.99 wt %, preferably about 90 wt % to about 99.99 wt % based on whole weight of the cosmetic composition according to the present invention. However, the ratio may depend on the above-mentioned formulations, a specific applying site thereof (face, neck, and the like), or a preferable applying amount thereof, so that it should not be understood that the above ratio is limited to the range of the present invention.

When the formulation of the present invention is paste, cream or gel, animal oil, vegetable oil, wax, paraffin, starch, tragacanth, cellulose derivative, polyethylene glycol, silicon, bentonite, silica, talc, zinc oxide, and the like may be used as a carrier component.

When the formulation of the present invention is powder or spray, lactose, talc, silica, aluminum hydroxide, calcium silicate, polyamide powder, and the like may be used as a carrier component, and especially, when it is spray, a propellant, such as chlorofluorohydrocarbon, propane/butane, or dimethylether, may be further included.

When the formulation of the present invention is solution or turbid fluid, solvent, solubilizer, or demulsifier, for example, water, ethanol, isopropanol, ethyl carbonate, ethyl acetate, benzyl alcohol, benzyl benzoate, propylene glycol, 1,3-butylglycol oil, glycerol fatty acid ester, polyethylene glycol, fatty acid of sorbitan, and the like may be used as a carrier component.

When the formulation of the present invention is suspension, liquid diluents, such as water, ethanol, or propylene glycol, suspension, such as ethoxylated isostearyl alcohol, polyoxyethylene sorbitol ester, or polyoxyethylene sorbitan ester, microcrystal cellulose, aluminum metahydroxide, bentonite, agar, tragacanth, and the like, may be used as a carrier component.

The cosmetic composition of the present invention may be used for cosmetics, such as skin toner, lotion, cream, essence lotion, pack, foundation lotion, color cosmetics, sunblock-cream, two-way cake, face powder, compact, makeup base, skin cover, eyeshadow, lipstick, lipgloss, lipfix, eyebrow pencil, skin lotion, and the like, and detergents, such as shampoo, soap, and the like.

The cosmetic composition of the present invention may further include fat materials, organic solvents, dissolvent, thickening agent, gelling agent, softener, antioxidants, suspension agent, stabilizer, foaming agent, flavoring agent, surfactant, water, ion-type or non-ion-type emulsifier, filler, sequestering agent, chelation agent, preserved agent, vitamins, blocker, moistening agent, essential oils, dye, pigment, hydrophilic or lipophilic activator, lipid vesicle, or adjurvants, such as any other components that are generally used for cosmetics and also in the field of cosmetology or dermatology. In addition, the above components may be introduced in such amount that is generally used in the field of dermatology.

Hereinafter, the present invention will be described in more detail with reference to Examples. It will be apparent by a person with ordinary skill in the art that Examples are only for describing the present invention, but the range of the present invention will not limited to Examples. Especially, it will be apparent by a person with ordinary skill in the art that alphaneoendorphin peptide will be described as an example in the following Examples of the present invention, but dynorphin is a material that is concerned in the same mechanism as alphaneoendorphin so that dynorphin is synthesized by the same method as that of alphaneoendorphin and has same or similar effect as new peptide derivative.

### Example 1

### Preparation of Caffeoyl (R₁, R₂, and R₅ are hydrogen, and R₃ and R₄ are hydroxy)-alphaneoendorphin Peptide Derivative

### 1.1: Synthesis of NH₂-protected Peptide-resin

Generally, peptide was synthesized by general solid phase peptide synthesis (SPPS) by using 9-fluorenylmethoxycarbonyl (Fmoc) as a protecting group of amino acid, and N-hydroxybenzotriazole (HOBt) and N,N′-dicyclohexylcarbodiimide (DCC) were used as a activator to extend amino acid residue [Reference Document: Wang C. Chan, Perter D. White, ‘Fmoc solid phase peptide synthesis,’ Oxford].

### 1.2: Synthesis of Caffeoyl-alphaneoendorphin Peptide

20% piperidine/NMP solution was added to NH₂-protected peptide (tyrosine-glycine-glycine-phenylalanine-leucine-arginine-lysine-tyrosine-proline-lysine)-resi n synthesized by the above-mentioned method to remove Fmoc binding to amino group, was washed with N-methyl-2-pyrrolidone (NMP) and dichloromethane (DCM), and then was coupling-reacted with 5 equivalent of caffeic acid (Available from Lancaster Company) at room temperature overnight. After completing the reaction, it was washed with NMP and DCM several times and then dried.

The dried caffeoyl-tripeptide-resin was reacted with mixed solution of trifluoroacetic acid:phenol:thioanisole:water:ethanedithiol (82.5:5:5:5:2.5 (v/v)) at room temperature for 2 to 3 hours to remove t-butyloxycarbonyl (Boc) and trityl(triphenylmethyl) group that are protecting groups of functional groups that are presented on side chain of amino acid residues consisting peptide; caffeoyl-decapeptide was removed from resin, and then the peptide was precipitated with cooled diethylether.

The obtained caffeoyl-tripeptide was purified through Reverse Phase High Performance Liquid Chromatography (Column: Gemini, C18 110A 250×21.2 mm) using acetonitrile and water containing 0.1% trifluoroacetic acid as a solvent.

Yield: 41%

### Experiment Example 1

### Physical Stability Test of Caffeoylalphaneoendorphin Peptide Derivative under the condition of Alternating Temperature

The physical stability under the condition of severe alternating temperature is an essential factor in the terms of the product property to be exposed to various external atmospheres in the case of cosmetics. The caffeoylalphaneoendorphin peptide derivative prepared from Example 1 has an increased stability to oxidation rather than that of caffeic acid in an aqueous solution. In order to confirm the increased physical stability, it was stored under the condition of alternating temperature for certain period time, and then its degradation degree was measured.

Firstly, 10,000 ppm concentration of caffeoylalphaneoendorphin peptide derivative aqueous solution was placed under the conditions of alternating temperatures, that is, low temperature (4° C.), room temperature (20° C.), and high temperature (40° C.) that were repeated at an interval of 8 hours, and then the physical stability was measured by the naked eye and HPLC Analysis for 30 days, 60 days, and 90 days.

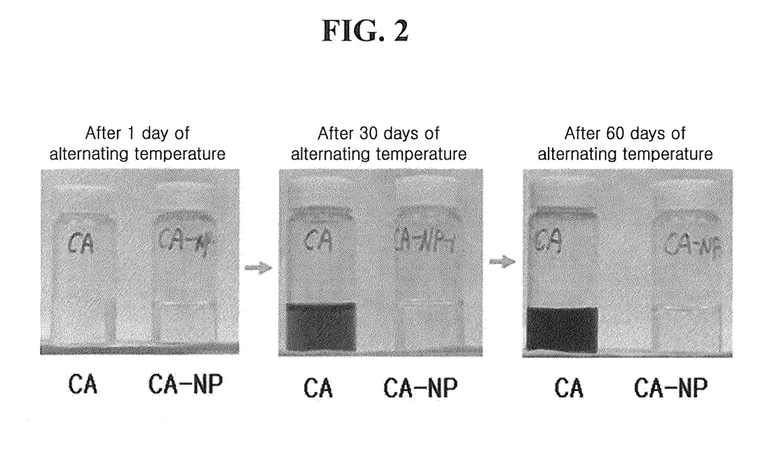

Firstly, a reduction in the level of deterioration of caffeoyl-alpha neo endorphin peptide derivative in the solution was observed through performing alternating temperature up to 60 days as compared with the deterioration of caffeic acid in the solution. As a result of observation, it was found that while the aqueous solution only with caffeic acid was changed to very dark brown from transparency so that it shown serious acidification, the transparency of the aqueous solution with caffeoyl-alphaneoendorphin peptide derivative was not changed so that there was no acidification (see FIG. 2).

Increased stabilities of peptides that had generally very low stabilities in aqueous solutions were measured through HPLC using a control of alphaneoendorphin. As a result, initial 100% content of caffeoylalphaneoendorphin peptide derivative was treated by alternating temperature for 0 day, 30 days, 60 days, and 90 days, and then their residual contents were 99±2%, 96±4%, 90±8%, and 81±19%, respectively, so that it was found that the physical stability of caffeoylalphaneoendorphin peptide derivative was very stably improved even under a serious alternating temperature condition. On the other hand, after the alphaneoendorphin, which was only peptide part, used for a control was treated by alternating temperature for 90 days, its residual content was 46±23% so that it was found that its physical stability was very decreased. For this reason, it was found that new caffeoyl-peptide derivative according to the present invention was suitable as a raw material in various industrial applications (For example: Cosmetics, Medicine, Pharmaceuticals) due to its improved physical stability.

### Experiment Example 2

### Stability Test of Caffeoylalphaneoendorphin Peptide Derivative to Peptidase

The caffeoylalphaneoendorphin peptide derivative prepared from Example 1 was a material that its caffeic acid has an anti-allergic effect as a main component for improving atopic dermatitis and alphaneoendorphin that is its peptide part effectively alleviates a specific itching due to an atopic dermatitis at the same time. However, the alphaneoendorphin that is a precursor as a peptide part was only constructs of amino acids, and was very weak in the action of proteolysis or peptidase so that it had a limitation of biological stability for applying as a cosmetics material.

The caffeoyl peptide derivative according to the present invention had an improved biological stability through the synthesis with caffeic acid. In order to confirm the improved stability to peptidase by synthesizing with caffeic acid, 10,000 ppm concentrations of alphaneoendorphin and caffeoylalphaneoendorphin peptide derivative were cultured with cell disruption solution of keratinocyte and fibroblast at 37° C. for 6 hours, and then the residual contents were measured through HPLC.

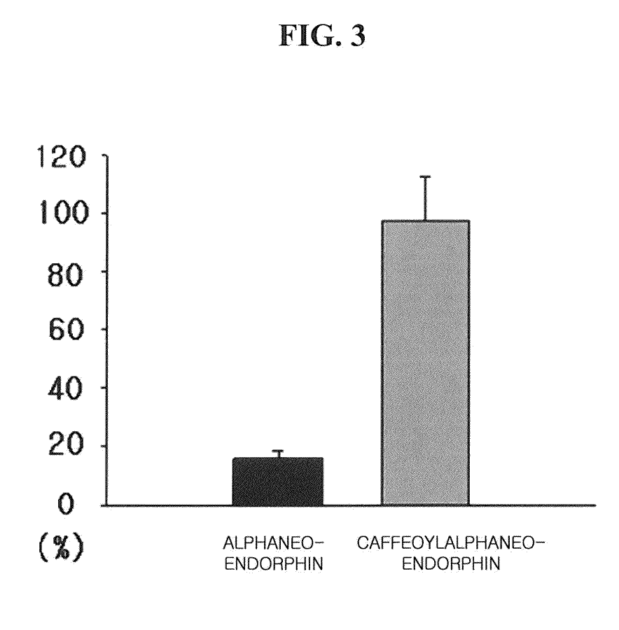

After culturing at 37° C. for 6 hours, the residual contents were analyzed, and the existing contents were compared and evaluated on a percentage (%) to the initial existing contents. As a result, while alphaneoendorphin that was not bound with caffeic acid was considerably degraded according to our expectations, caffeoylalphaneoendorphin peptide derivative was almost not degraded so that the existing contents were increased by 6.1±1.3 times and 5.9±2.1 times, respectively from the degradation of peptidase derived from the cell disruption solution of keratonocyte and fibroblast. Especially, FIG. 3 is a graph showing an experiment result of cell disruption solution of keratinocyte, in which a left bar indicates an experiment result of alphaneoendorphin, and a right bar indicates an experiment result of caffeoylalphaneoendorphin peptide derivative.

### Experiment Example 3

### Skin Irritation Safety Test of Caffeoylalphaneoendorphin Peptide Derivative

In order to confirm the safety of caffeoyl peptide derivative according to the present invention to a human body skin when applying besides a skin that is an expected first human body application method, a topical toxicity skin irritation safety that is pre-clinical test method of pharmaceuticals was verified. The safety verification was performed by asking to Korea Research Institute of Chemical Technology that is an approved GLP Certificate Authority; the maximum application concentration range to skin irritation during topical toxicity was set at 10 mg/ml concentration, and then it was tested that irritant to a skin at the above concentration was not existed. For the above test, at least 6 of young and healthy white rabbits (New Zealand White-Rabbit, 2.0˜3.0 kg) were used. After selecting only healthy rabbits through observing a general symptom every day for 5 days during an accommodating period from an obtaining day, hairs of the abdominal regions of the selected rabbits was shaved away and then 6 rabbits with normal skins were used for test. And, for dividing a group, when the hairs of the rabbits were shaved away, and then the rabbits do not have abnormality on their skins, the rabbits were selected and used. An keeping environment was simply as follows: the test was performed in a rabbit keeping room in the institute by setting the temperature of 22° C., the relative humidity of 50%, a number of ventilation of 10˜15 number/hr, lighting of 12 h lights-on/12 h lights-off (08:00˜20:00), an intensity of illumination of 150˜300 Lux. For monitoring of keeping environment, whole humidity in an animal room was measured by an auto-measurer and environment conditions, such as, an intensity of illumination were regularly measured during a test period. As a result of environment measurement in an animal room, there was no change to affect to the test. The animals were kept individually in a stainless keeping box (380×480×300 mm, JungDo Industry) during accommodation, inspection, and observation periods. Feeds that was feeds for Rabbits, experiment animals, supplied from Cargillagripurina Cor. (Chonbuk, Gunsan-si Soyong dong 56-4) was freely taken by the animals, and water that was a filtered water through UV irradiation was freely taken by using a water bottle. In order to confirm the contaminant in the feeds and water, the contaminant of feeds was confirmed by receiving the feed report card according to regular test of manufacturer from a feeds supplier, and the contaminant of water was confirmed through the regular test according to applicable standard working water procedure (SOPs) of the institute.

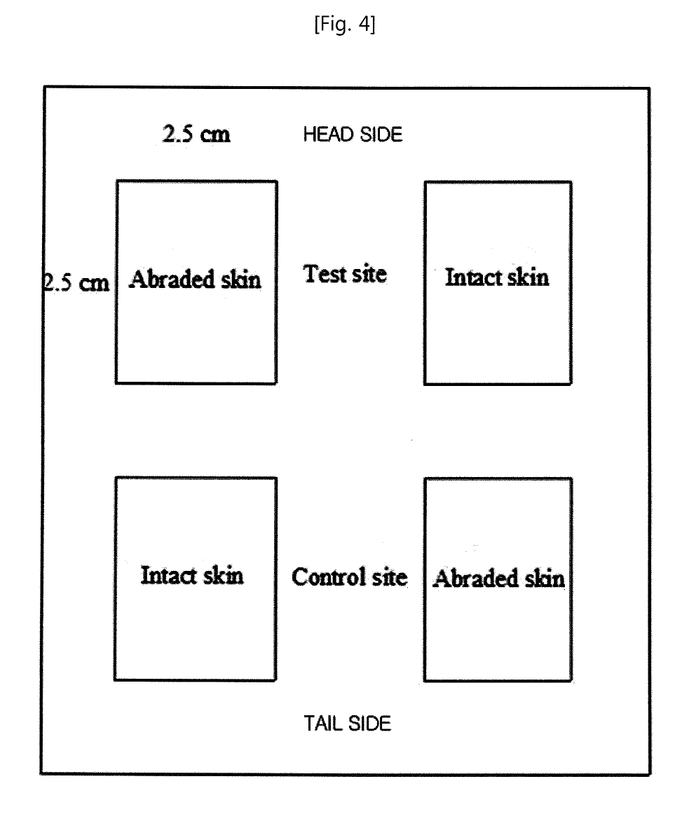

The caffeoylalphaneoendorphin peptide derivative that was a test material prepared from Example 1 was dissolved in a concentration of 10 mg/ml (10,000 ppm), and then 0.5 ml per each animal was used by using the following method. That is, after shaving away before about 24 hours of application of test material, as shown in FIG. 4, two parts of normal skin (Intact Skin) and two parts of damaged skin (Abraded Skin) with the size of (2.5×2.5) cm were divided and then the test materials and solvent were applied thereon. The abraded skins were made to have a scratch without blood by using a needle end of syringe so that the horny layer of the skin was damaged, but the corium of the skin was not damaged. After the test materials were applied to one site of abraded skins and one site of intact skins, the sites were occlusive-patched with non-irritative tape and Coban for 24 hours. Control regions were applied with sterilized gauze wetted with sterilized distilled water by using the same method as the above (see FIG. 4).

In order to observe as to whether skin irritation was occurred, a change of general symptoms and as to whether animals were killed were observed during 72 hours after completing the application of test materials as the observation of general symptoms. In addition, the body weights per individual were measured immediately before applying the test materials and at 72 hours after applying the test materials. In order to specifically evaluate the skin irritation, whether or not the skin irritation, such as erythema, thick skin, edema, and the like were observed at 24 hours and 72 hours after completing the application of test materials. The evaluation of skin response and decision of irritant were performed based on the evaluation table of skin response as disclosed in the following Table 1. The decision of irritant content to the result was performed according to a calculation method of P.I.I. (Primary Irritation Index) by Draize, which is well used, generally (see Table 2).

TABLE 1
Evaluation table of skin response
(1) Erythema and crust formation

| | |
|---|---|
| Absolutely no erythema | 0 |
| Very light erythema (barely identifiable level with the naked eye) | 1 |
| Obvious erythema | 2 |
| A little serious erythema | 3 |
| Serious erythema (carrot color) and crust formation | 4 |
| Sum of total possible levels | 4 |

(2) Edema Formation

| | |
|---|---|
| Absolutely no edema | 0 |
| Very light edema (Identifiable level with the naked eye) | 1 |
| Mild edema (Swelling distinctly up so that marginal zone | 2 |
| can be distinguished) | |
| Serious edema (In the case of swelling up to about 1 mm) | 3 |
| Heavy edema (In the case of swelling up at least 1 mm and | 4 |
| extended to the outside of exposure site) | |
| Sum of total possible levels | 4 |

TABLE 2
Irritant Evaluation by P.I.I.

| Degree (P.I.I) | Section |
|---|---|
| 0.0~0.5 | None irritant |
| 0.6~2.0 | Mild irritant |
| 2.1~5.0 | Moderate irritant |
| 5.1~8.0 | Severe irritant |

As a result of performing a skin irritation test of topical toxicity with the above-mentioned method, it was verified that 10 mg/ml concentration of caffeoyl-alphaneoendorphin peptide derivative has no toxicity as disclosed in the following Table 3 to Table 5.

A mortality and general symptoms generated during the test procedure for verifying the skin irritation safety of topical toxicity were shown in Table 3, and there were no death or unusual symptoms related to the application of test materials during the test period.

The changes of body weights were shown in Table 4, and as the results of weighing the body weights, the body weights of all of animals were normally increased.

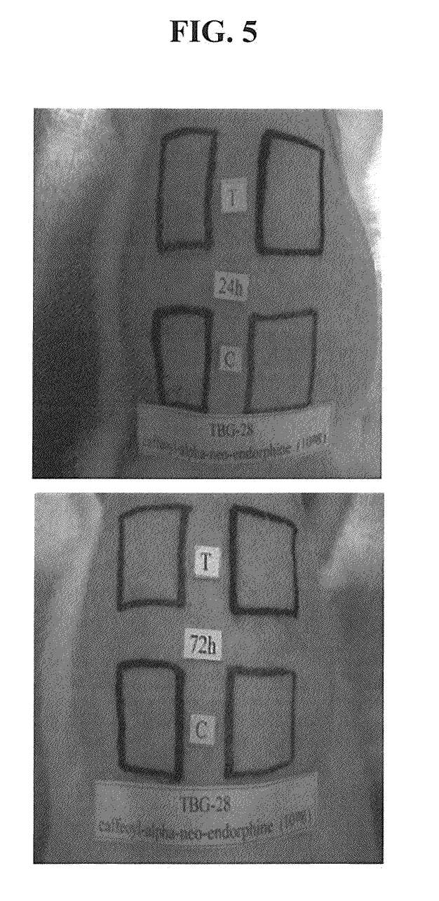

In order to evaluate the skin irritation safety, the results of observing the application sites were shown in Table 5. That is, as the result of observing the skin response at 24 and 72 hours after completing the exposures of test materials, Primary Irritation Index (P.I.I) was evaluated as “0.0” because there were no the skin responses, such as erythema, edema, and the like (see FIG. 5). From the results as mentioned above, it was found that 10 mg/ml concentration of caffeoylalphaneoendorphin peptide derivative was none irritant material that was safe for skin based on the standard of Table 2 when applying it over NZW Rabbit skins.

TABLE 3
Mortality and clinical signs

| | No. | | | | | |
|---|---|---|---|---|---|---|
| | (Animal | (Hours after application) | | | | |
| (Sex) | number) | 0 | 24 | 48 | 72 | (Mortality) |
| (Female) | 1 | — | — | — | — | 0/6^{a} |
| | 2 | — | — | — | — | |
| | 3 | — | — | — | — | |
| | 4 | — | — | — | — | |
| | 5 | — | — | — | — | |
| | 6 | — | — | — | — | |

—: Normal
^{a}Number of dead animals/Number of total animals)

TABLE 4
Body weights

| No. | | |
|---|---|---|
| (Animal | (Hours after application) (g) | (Gains) |

| (Sex) | number) | 0 | 72 | (g) |
|---|---|---|---|---|
| (Female) | 1 | 2408.4 | 2496.7 | 88.3 |
| | 2 | 2478.1 | 2611.7 | 133.6 |
| | 3 | 2347.3 | 2454.4 | 107.1 |
| | 4 | 2237.2 | 2313.4 | 76.2 |
| | 5 | 2511.9 | 2631.4 | 119.5 |
| | 6 | 2684.9 | 2788.0 | 103.1 |

TABLE 5
Evaluation of skin irritation

| | Sites | | | | | | | |
|---|---|---|---|---|---|---|---|---|
| | Control sites | | | | | | | |
| | Change | | | | | | | |
| | Erythema & Eschar | | | | Edema | | | |
| | Intact | | Abraded | | Intact | | Abraded | |
| | 24 | 72 | 24 | 72 | 24 | 72 | 24 | 72 |
| Phases¹ | Hour | Hour | Hour | Hour | Hour | Hour | Hour | Hour |
| 1 | 0 | 0 | 0 | 0 | 0 | 0 | 0 | 0 |
| 2 | 0 | 0 | 0 | 0 | 0 | 0 | 0 | 0 |
| 3 | 0 | 0 | 0 | 0 | 0 | 0 | 0 | 0 |
| 4 | 0 | 0 | 0 | 0 | 0 | 0 | 0 | 0 |
| 5 | 0 | 0 | 0 | 0 | 0 | 0 | 0 | 0 |
| 6 | 0 | 0 | 0 | 0 | 0 | 0 | 0 | 0 |
| Sum | 0 | 0 | 0 | 0 | 0 | 0 | 0 | 0 |
| Average | 0 | 0 | 0 | 0 | 0 | 0 | 0 | 0 |
| Sum of | 0.0 | | | | | | | |
| Averages | | | | | | | | |
| P.I.I² | 0.0 | | | | | | | |
| | Sites | | | | | | | |
| | Test sites | | | | | | | |
| | Change | | | | | | | |
| | Erythema & Eschar | | | | Edema | | | |
| | Intact | | Abraded | | Intact | | Abraded | |
| | 24 | 72 | 24 | 72 | 24 | 72 | 24 | 72 |
| Phases¹ | Hour | Hour | Hour | Hour | Hour | Hour | Hour | Hour |
| 1 | 0 | 0 | 0 | 0 | 0 | 0 | 0 | 0 |
| 2 | 0 | 0 | 0 | 0 | 0 | 0 | 0 | 0 |
| 3 | 0 | 0 | 0 | 0 | 0 | 0 | 0 | 0 |
| 4 | 0 | 0 | 0 | 0 | 0 | 0 | 0 | 0 |
| 5 | 0 | 0 | 0 | 0 | 0 | 0 | 0 | 0 |
| 6 | 0 | 0 | 0 | 0 | 0 | 0 | 0 | 0 |
| Sum | 0 | 0 | 0 | 0 | 0 | 0 | 0 | 0 |
| Average | 0 | 0 | 0 | 0 | 0 | 0 | 0 | 0 |

| | |
|---|---|
| Sum of | 0.0 |
| Averages | |
| P.I.I² | 0.0 |

¹Time after topical application
²P.I.I(Primary Irritation Index) = (Sum or mean at 24 and 72 hrs)/4

### Experiment Example 4

### Effectivity Evaluation of Caffeoylalphaneoendorphin Peptide Derivative through Rat Mite Atopic Animal Model

In order to confirm an atopic dermatitis improvement effect of caffeoylalphaneoendorphin peptide derivative prepared from Example 1, firstly Balb/c mice were induced to be taken with an atopic dermatitis. Firstly, 20 ul of 2% DNCB was applied to ear to boost an immune response, and then after 1 week, 20 ul of 2% DNCB and 15 ul of dust mites (10 mg/ml) were again treated in turn once a week. After proceeding the induction for 3 weeks, IgE level was confirmed in serum, and then the mice were divided into total 5 groups. While the induction was performed together every day for 3 weeks for confirming the improvement effectivity, caffeoylalphaneoendorphin peptide derivative was dissolved in 70% EtOH to be 1 mg/ml concentration, and then applied to each ear (pep.400: caffeoylalphaneoendorphin peptide derivative 400 ug/each ear, pep.800: caffeoylalphaneoendorphin 800 ug/each ear); and the same amount of 70% EtOH was treated as a control. During the induction period, DNCB or dust mites was treated, and then after one day, ear thicknesses were measured to confirm the change of ear thicknesses and ear symptoms, and then reported. After finally treating for 3 weeks, the mice were sacrificed, the thickness changes and pathologic changes of ears that were an atopic dermatitis inducement sites were examined.

4.1. Apparent Effectivity

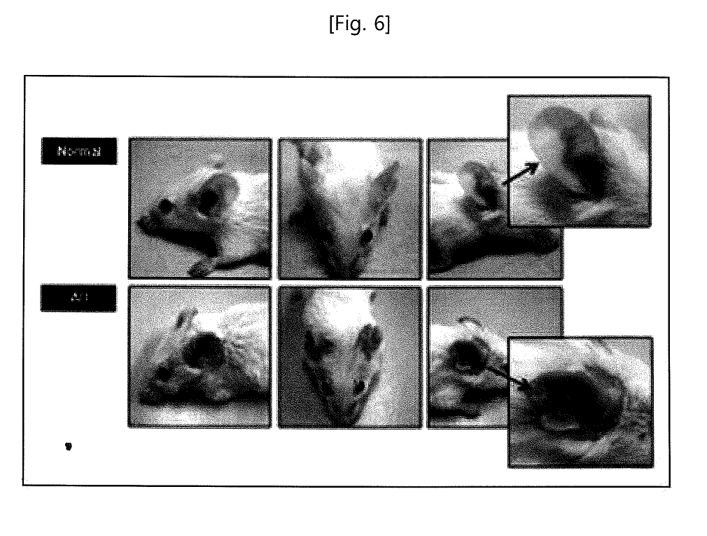

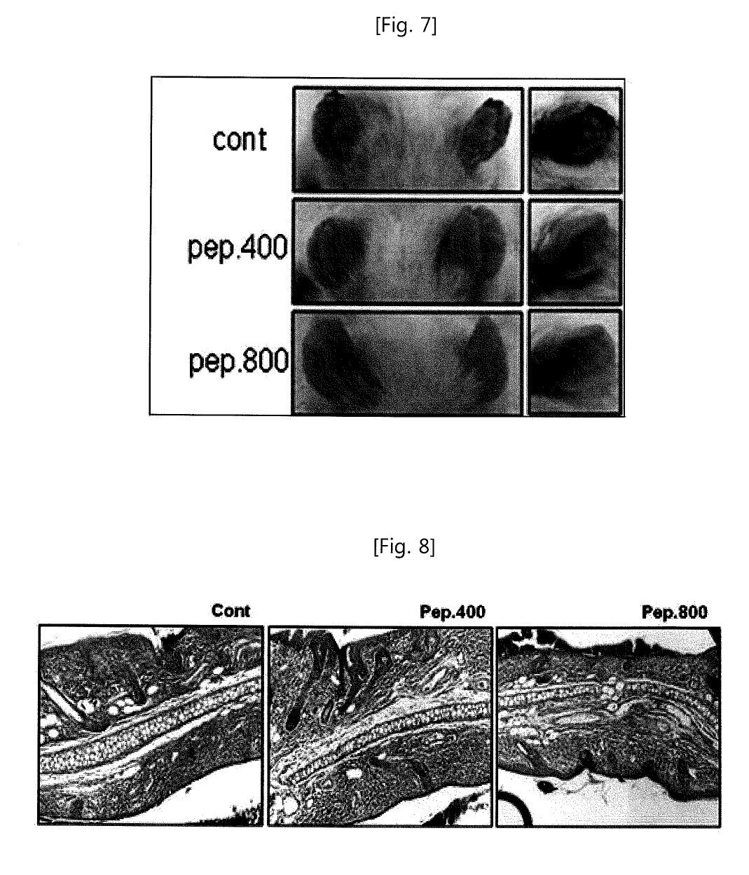

First of all, an apparent change of ear was confirmed in order to confirm the effectivity of the treatment materials. As shown in FIG. 6, it was found that in the case of the atopic dermatitis inducement for total 6 weeks, the ear thickness was considerably increased, a considerable amount of dead skin cells were produced, and the ear had big swelling due to the inflammatory response as shown in the control group. In addition, it was also found that there were many scabs and the ear shape was changed due to dry and cell necrosis. On the other hand, when treating caffeoyl-alphaneoendorphin peptide derivative, all visible symptoms were considerably moderated as compared with the control, generally so that it shown a healthy condition. Especially, it was found that the thickness and dead skin cell of ear, the degree of erythema, and the degree of hardening were considerably decreased as compared with the control group (see FIG. 7).

4.2. Pathological Comparison of Ear

Because the increases of the thickness of ear that had an atopic dermatitis symptom means that a lot of immune cells were moved to the ear due to the inflammatory response, the immune cells that were presented in the ear were confirmed through H&E staining method. At the staining process, hematoxylin stains nucleus to express it as dark blue or purple color dot and eosin stains cytoplasm to express it as red violet color. For the staining, ears of each group were cut, fixed with paraffin, and then cross-sections of ears were cut for staining. As shown in FIG. 8, it was found that when comparing the ears of each test group, a number of the immune cells stained with dark blue or purple color in the group of pep. 800 (caffeoyl-alphaneoendorphin peptide derivative 800 ug/each ear) was significantly decreased as compared with the control group (see FIG. 8).

4.3. Change of IgE Concentration in Serum

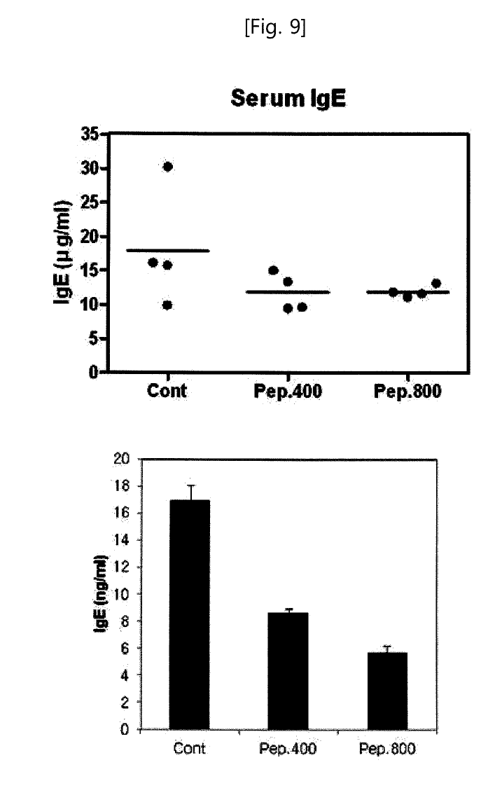

Next, the effect on IgE concentration in serum that is being used as a biggest marker for an atopic dermatitis was confirmed. Firstly, after preparing serum, total IgE concentration that is allergen non-specific was confirmed through ELISA, and then the results were shown in FIG. 9. It was found that in the case of total IgE, the experiment groups of 400 ug (per each ear) and 800 ug (per each ear) treatment of caffeoylalphaneoendorphin peptide derivative decreased IgE concentration in serum as compared with the control group (see Top diagram of FIG. 9).

Based on the fact that IgE concentration was decreased in serum when continually treating caffeoylalphaneoendorphin peptide derivative, we tried to confirm as to whether the above two materials directly affect to B cell that is a major cell for secreting IgE (B cell intrinsic effect). To achieve this, B cell was extracted from lymphnode of each mouse, the extracted B cell was stimulated with IL-4 and LPS that can help to secrete IgE, and then IgE concentration secreted from the B cell was measured with ELISA. As shown in the bottle diagram of FIG. 9, it was found that IgE in culture medium secreted from the B cell was decreased, and its suppression ability had strengthened with increasing the concentration. That is, it was found that when continually treating caffeoylalphaneoendorphin peptide derivative, it directly affects to B cell so that IgE secretion was suppressed.

4.4. Atopic Dermatitis Inducible Cytokine Expression Amount Comparison in Lymphnode CD4+ T cell

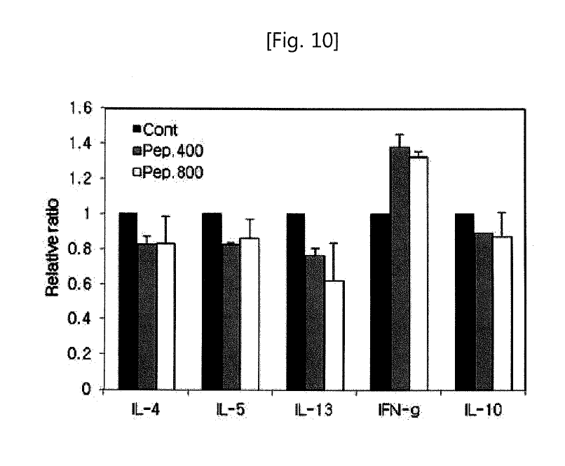

It is well known that in the first stage of atopic dermatitis, cytokine, IL-4, IL-5, IL-13, and the like, that are expressed in Th2 cell are very important, but in order to maintain the progressed atopic dermatitis, Th1 type cytokine, IFNg, IL-12, and the like that are expressed in Th1 cell play an important role. Since the atopic dermatitis is an inflammatory diseases, cytokine, IL-17 and TNFα, related to an inflammation induction play also an important role for an onset of atopic dermatitis. Therefore, it was confirmed through a real-time PCR what are the effects of the continuous treatment of caffeoyl-alphaneoendorphin peptide derivative on the expression of cytokines that play an important role for an onset of the atopic dermatitis. To achieve this, CD4+ T cell was extracted from lymphnodes of mice of each group, and also ears were cut to confirm the cytokines expression amount in mRNA level in total cell. Firstly, in the case of CD4+ T cell, when comparing the expression amounts of IL-4, IL-5, IL-13, IL-10 and IFN-γ, while the expression amounts of IFN-γ and IL-10 didn't changed very much, the expression amounts of IL-4, IL-5, and IL-13 were decreased and especially the expression amount of IL-13 was significantly decreased (see FIG. 10).

4.5. Atopic Dermatitis Inducible Cytokine Expression Amount Comparison in the Whole Ear Cells

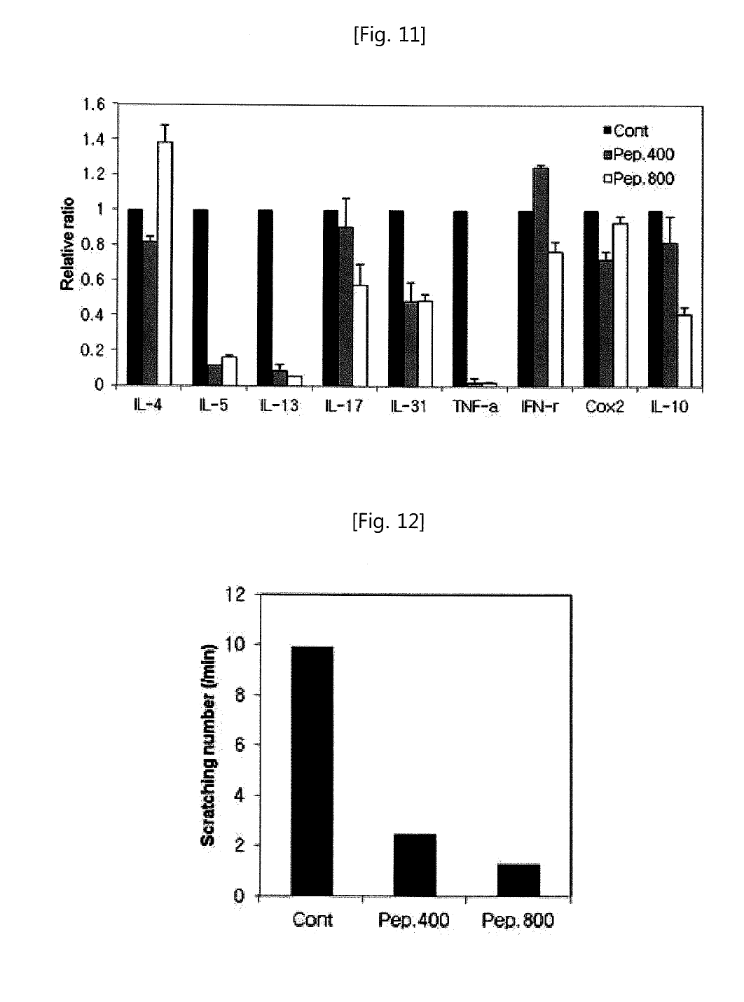

Next, when comparing the expression amounts of cytokines in the whole ear cells isolated from the ear, the cytokines in the whole ear cells were significantly decreased generally as compared with the cytokines expressed in Lymphnode T cell. That is, all the expressions of IL-4, IL-5, IL-13, IL-17, IL-31, TNF-α, IFN-γ, and IL-10 were significantly decreased in the groups treated with caffeoylalphaneoendorphin peptide derivative. In addition, it was found that the decrease phenomenon depended on the concentration because the expressions of IL-13, IL-17, IFN-γ, and IL-10 were decreased even more with increasing the treatment concentration of caffeoyl-alphaneoendorphin peptide derivative (see FIG. 11). From the above experiment results, it is though that the immune response can be controlled by decreasing the expressions of cytokines (IL-4, IL-5, IL-13, IL-31, IL-10) of Th2 type and inflammatory cytokines (TNF-α, IL-17) through the continuous treatment of caffeoylalphaneoendorphin peptide derivative. Especially, it is thought that since it is known that IL-4, IL-5, and IL-13 play an important role for secreting IgE by B cell, IL-4, IL-5, and IL-13 are concerned to the above process.

4.6. Decrease Effect of Caffeoyl Alpha Neo Endorphin Peptide Derivative on Itching

The biggest symptom property of atopic dermatitis is a scratching due to a serious itching. The continuous scratching causes an abrasion on a skin, more deteriorates an inflammatory response, and more deteriorates an atopic symptom. Therefore, the suppression of the scratching may be possible to use as an effective treatment method for alleviating the atopic symptom.

Especially, caffeoyl peptide derivative according to the present invention is a combination with alphaneoendorphin that can alleviate the itching by competing the neurotransmission route of itching. For this reason, we tried to confirm as to whether the combination can reduce the number of scratching when actually treating the combination to mice. To achieve this, the cage of each group was recorded on videotape for about 10 minutes using a camcorder, and then total number of scratching was measured per each group.

As shown in Table 6, when summing total numbers of scratching of mice per groups, while the control was averagely 10 times per one minute, the group treated with 400 ug of caffeoylalphaneoendorphin peptide derivative was 2.5 times/minute and the group treated with 800 ug of caffeoylalphaneoendorphin peptide derivative was 1.3 times/minute, so that it was found that the number of scratching in the treatment group was significantly decreased as compared with the control group. As the previous cytokine result, it was found that the itching in the mice inducible atopic dermatitis model of caffeoylalphaneoendorphin peptide derivative was alleviated so that the number of scratching was reduced, and the number of scratching was more effectively decreased with increasing the concentration of caffeoyl-alphaneoendorphine peptide derivative (see FIG. 12).

TABLE 6

| | Number of | | |
|---|---|---|---|
| | Scratching | Hour | /Min |
| Cont. | 129 | 13 | 9.923077 |
| Pep. 400 | 27 | 11 | 2.454545 |
| Pep. 800 | 14 | 11 | 1.272727 |

### Formulation Example 1

### Preparation of Skin Lotion Including Caffeoyl Peptide Derivative

A skin lotion including caffeoyl peptide derivative prepared from Example 1 was prepared in the components and contents as disclosed in the following Table 7.

Specifically, the components, 2, 3, 4, and 8 were added to the component, 11 in order, stirred, and dissolved (Solution 1). On the other hand, after the component, 5 was heated at about 60° C. and dissolved, the component, 10 was added thereto (Solution 2). And then, the solution 2 was added to the solution 1, and finally the components, 1, 6, 7, and 9 were added, fully stirred, and then aged to prepare the skin lotion.

TABLE 7

| No. | Component | Content (wt %) |
|---|---|---|
| 1 | Aqueous Solution of Caffeoyl Peptide | 1.0 |
| | Derivative (100 ppm) of Example 1 | |
| 2 | Glycerin | 3.0 |
| 3 | Butyleneglycol | 2.0 |
| 4 | Propyleneglycol | 2.0 |
| 5 | Polyoxyethylene Hydrogenated Castor Oil | 1.5 |
| 6 | Ethanol | 8.5 |
| 7 | Triethanolamine | 0.1 |
| 8 | Preservative | Very Small Amount |
| 9 | Pigment | Very Small Amount |
| 10 | Flavoring | Very Small Amount |
| 11 | Purified Water | Residual Quantity |

### Formulation Example 2

### Preparation of Nutrition Lotion Including Caffeoyl Peptide Derivative

A nutrition lotion including caffeoyl peptide derivative prepared from Example 1 was prepared in the components and contents as disclosed in the following Table 8.

Specifically, after the components, 10, 11, 13, and 16 were heated at 80˜85° C. while mixing and stirring; the mixture was added to a preparing part and then emulsified. And then, the components, 2, 3, 4, 5, 6, 7, 8, and 12 were heated at 80˜85° C. to dissolve, and then added to emulsify. After completing the emulsification, while stirring with a stirrer, the mixture was cooled to 50° C.; then the component, 15 was added and cooled to 45° C.; the component, 14 was added; the component, 1 was added at 35° C. and then cooled to 25° C.; and then aged to prepare the nutrition lotion.

TABLE 8

| No. | Component | Content (wt %) |
|---|---|---|
| 1 | Aqueous Solution of Caffeoyl Peptide | 1.0 |
| | Derivative (100 ppm) of Example 1 | |
| 2 | Wax | 1.0 |
| 3 | Polysolvate 60 | 2.0 |
| 4 | Sorbitan Sesquioleate | 0.5 |
| 5 | Liquid Paraffin | 9.5 |
| 6 | Sorbitan Stearate | 1.0 |
| 7 | Lipophilic Glyceryl Monostearate | 0.2 |
| 8 | Stearic Acid | 1.5 |
| 9 | Glyceryl Stearate | 1.5 |
| 10 | Propylene Glycol | 1.0 |
| 11 | Carboxy Polymer | 2.5 |
| 12 | Triethanolamine | 0.1 |
| 13 | Preservative | 0.2 |
| 14 | Pigment | Very Small Amount |
| 15 | Flavoring | Very Small Amount |
| 16 | Purified Water | Residual Quantity |

A caffeoyl endorphin peptide derivative according to the present invention is safe for skin; also resistant to degradation occurred by peptidase, and the like when applying it over the skin; and has an excellent stability against temperature change, and the like so that it can be applied to cosmetic materials for anti-inflammatory use, such as atopic dermatitis treatment, and the like.

While the present invention has been particularly shown and described with reference to exemplary embodiments thereof, it will be understood by those of ordinary skill in the art that various changes in form and details may be made therein without departing from the spirit and scope of the present invention as defined by the following claims.

1. A peptide derivative represented by the following Chemical Formula 1: 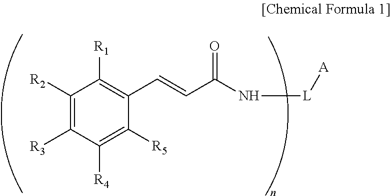
where,
R₁, R₄, and R₅ are independently hydrogen, hydroxy, C₁-C₆ alkyl group, C₁-C₆ alkoxy group, cyano, halo, or amino, respectively;
R₂ and R₃ are hydroxy;
n is 1;
L is either absent or a linking group; and
A is peptide or fragment thereof having the sequence Tyr-Gly-Gly-Phe-Leu-Arg-Arg-Ile-Arg-Pro-Lys-Leu-Lys (YGGFLRRIRPKLK).

2-7. (canceled)

8. The peptide derivative according to claim 1, further comprising at least one of a adjuvant, solution, suspension, turbid fluid, paste, gel, cream, powder, and spray.

9. The peptide derivative according to claim 8, further comprising at least one of a stabilizer, solubilizer, vitamin, pigment, and flavoring.

10. The peptide derivative according to claim 8, further comprising at least one of an animal oil, vegetable oil, paraffin, starch, tragacanth, cellulose derivative, polyethylene glycol, silicon, bentonite, silica, talc, and zinc oxide.

11. The peptide derivative according to claim 8, further comprising at least one of a lactose, talc, silica, aluminum hydroxide, calcium silicate, and polyamide powder.

12. The peptide derivative according to claim 8, further comprising a propellant.

13. The peptide derivative according to claim 12, further comprising at least one of a chlorofluorohydrocarbon, propane, butene, and dimethylether.

14. The peptide derivative according to claim 8, further comprising at least one of water, ethanol, isopropanol, ethyl carbonate, ethyl acetate, benzyl alcohol, benzyl benzoate, propylene glycol, 1,3-butylglycol oil, glycerol fatty acid ester, polyethylene glycol, and fatty acid of sorbitan.

15. The peptide derivative according to claim 8, further comprising at least one of water, ethanol, propylene glycol, ethoxylated isostearyl alcohol, polyoxyethylene sorbitol ester, polyoxyethylene sorbitan ester, microcrystal cellulose, aluminum metahydroxide, bentonite, agar, and tragacanth.

16. The peptide derivative according to claim 1, further comprising at least one of a fat material, organic solvent, dissolvent, thickening agent, gelling agent, softener, antioxidant, suspension agent, stabilizer, foaming agent, flavoring agent, preserved agent, vitamin, blocker, moistening agent, essential oil, dye, pigment, hydrophilic activator, lipophilic activator, lipid vesicle, and adjuvant.

17. The peptide derivative according to claim 1, further comprising a carrier present in an amount of about 1 wt % to about 99.99 wt %.

18. The peptide derivative according to claim 1, wherein the carrier is present in the amount of about 90 wt % to about 99.99 wt %.

19. A method comprising administering to a subject a caffeoyl peptide derivative represented by the Chemical Formula 1: 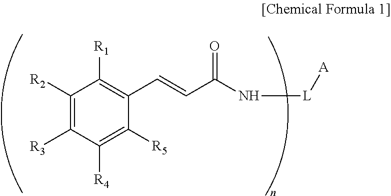
where,
R₁, R₄, and R₅ are independently hydrogen, hydroxy, C₁-C₆ alkyl group, C₁-C₆ alkoxy group, cyano, halo, or amino, respectively;
R₂ and R₃ are hydroxy;
n is 1;
L is either absent or a linking group; and
A is peptide or fragment thereof having the sequence Tyr-Gly-Gly-Phe-Leu-Arg-Arg-Ile-Arg-Pro-Lys-Leu-Lys (YGGFLRRIRPKLK).

20. The method according to claim 19, wherein the administering comprises at least one of a topical, oral, and intravenous administration.

21. The method according to claim 19, wherein the administering is to treat or prevent an atopic dermatitis.

22. The method according to claim 19, wherein the administering is to prevent or alleviate skin itching.

23. The method according to claim 19, wherein the administering is to suppress or reduce expression of an inflammatory factor.

24. The method according to claim 23, wherein the inflammatory factor comprises at least one of IL-4, IL-5, IL-13, IL-31, IL-10, TNFα, and IL-17.

25. The method according to claim 19, wherein the administering further comprises administering at least one of a adjuvant, solution, suspension, turbid fluid, paste, gel, cream, powder, and spray.

26. The method according to claim 19, wherein the peptide derivative is administered in a cosmetic composition.
